SEQUENCE LISTING (10 sequences)

SEQ ID NO: 1 - DNA, 22 bp, synthetic construct
  source 1..22: mol_type = other DNA
atgtccatgctgcaagcaatgt

SEQ ID NO: 2 - DNA, 22 bp, synthetic construct
  source 1..22: mol_type = other DNA
gcacacctgaaggaagaccaac

SEQ ID NO: 3 - DNA, 19 bp, synthetic construct
  source 1..19: mol_type = other DNA
atcacaccctggtgtagac

SEQ ID NO: 4 - DNA, 22 bp, synthetic construct
  source 1..22: mol_type = other DNA
ccataggagcagctgaaatata

SEQ ID NO: 5 - DNA, 19 bp, synthetic construct
  source 1..19: mol_type = other DNA
agcagctgaagccataatc

SEQ ID NO: 6 - DNA, 36 bp, synthetic construct
  source 1..36: mol_type = other DNA
tatgaaaactggtaaaagattatggctcagctccta

SEQ ID NO: 7 - DNA, 34 bp, synthetic construct
  source 1..34: mol_type = other DNA
tatgaaaactggttatattttcagctgcctccta

SEQ ID NO: 8 - DNA, 630 bp, synthetic construct
  source 1..630: mol_type = other DNA
caacatggaaggtagctcctgccctggctgctggctgtacagctgtactaaaaccatctgaattggcttccgtgtaagtttaacatgttaacttgttaatgtcatacccatgctagttgcaatgacatttgattttaaaatgttgtggcatgtccatgctgcaagcaatgtaatttgaaatctctctctatcattaattaccaggacttgtttggagcttgctgatgtgtgtaaagaggttggtcttccttcaggtgtgctaaacatagtgactggattaggttctgaagccggtgctcctttgtcatcacaccctggtgtagacaaggtacagctattcctcctgtaatcatgtataccccatcaatggaaatgatattcctctcaatacatggtttatgttttctgttaggttgcatttactgggagttatgaaactggtatatcagctgctcctatggttaaggtttgtttccaaatttctgtggatattttttgttctctttctactaactctctattatcaattctcaatgttgtccttttcttttaactcctttactttttagaattgtgatcaagacactttgagcatcattctagtagccagttctatcctgtttcttacct

SEQ ID NO: 9 - DNA, 606 bp, synthetic construct
  source 1..606: mol_type = other DNA
ttggagcttggtgatgtgtgtaaagaggttggtcttccttcaggtgtgctaaacatagtgactggattaggttctgaagccggtgctcctttgtcatcacaccctggtgtagacaaggtacagctattcctcctgtaatcatgtataccccatcaatggaaatgatattcctctcaatacatggtttatgttttctgttaggttgcatttactgggagttatgaaactggtatatatttcagctgctcctatggttaaggtttgtttccaaatttctgtggatattttttgttctctttctactaactctctattatcaattctcaatgttgtccttttcttttaactcctttactttttagaattgtgatcaagacactttgagcatcattctagtagccagttctatcctgtttcttacctttttatggttcgtcttttcttgacagcctgtttcactggaacttggtggaaaaagtcctatagtggtgtttgatgatgttgatgttgaaaaaggtacatgccacttgctatgattaactaattctgaagtgcgggactttgtaaagcacttaactgagctggatgctagacccccaaaagcca

SEQ ID NO: 10 - DNA, 616 bp, synthetic construct
  source 1..616: mol_type = other DNA
ttggagctttggtgatgtgtgtaaagaggttggtcttccttcaggtgtgctaaacatagtgactggattaggttctgaagccggtgctcctttgtcatcacaccctggtgtagacaaggtacagctattcctcctgtaatcatgtataccccatcaatggaaatgatatttcctctcaatacatggtttatgttttctgttaggttgcatttactgggagttatgaaactggtaaaaagattatggcttcagctgctcctatggttaaggtttgtttccaaatttctgtggatattttttgttctctttctactaactctctattatcaattctcaatgttgtccttttcttttaactcctttactttttagaattgtgatcaagacactttgagcatcattctagtagccagttctatcctgtttcttacctttttatggttcgtcttttcttgacagcctgtttcactggaacttgggtggaaaaagtcctatagtggtgtttgatgatgttgatgttgaaaaaggtacatgccacttgctatgattaactaattctgaagtgcgggactttgtaaagcacttaactgagctggatgctagaccccccaaaagc

(19) United States
(12) Patent Application Publication
(10) Pub. No.: US 2024/0417815 A1
(43) Pub. Date: Dec. 19, 2024

(54) PRIMER SET, KIT AND METHOD FOR QUALITATIVE AND QUANTITATIVE DETECTION OF SCENTED RICE

(71) Applicants: Nanjing University of Finance and Economics, Nanjing (CN); Central South University of Forestry and Technology, Changsha (CN)

(72) Inventors: Yong Fang, Nanjing (CN); Xinyue Li, Ya' an (CN); Peng Li, Nanjing (CN); Changrui Xing, Nanjing (CN); Fei Pei, Nanjing (CN); Qinlu Lin, Changsha (CN)

(21) Appl. No.: 18/485,359

(22) Filed: Oct. 12, 2023

(30) Foreign Application Priority Data
Jun. 15, 2023 (CN) 2023107063937

(51) Int. Cl. C12Q 1/6895; C12Q 1/6851

(52) U.S. Cl. CPC C12Q 1/6895; C12Q 1/6851; C12Q 2600/13

(57) ABSTRACT

A qualitative and quantitative detection primer set, a kit and a detection method for scented rice are provided. The scented rice and non-scented rice are qualitatively analyzed by fluorescence quantitative polymerase chain reaction (PCR), and the scented rice with different adulteration ratios is calculated by a 2^{-ΔΔCT} method, so that the scented rice and the non-scented rice of different varieties can be accurately identified. Primers are designed for specific fragments of the scented rice and the non-scented rice respectively based on the principle of amplification-refractory mutation system (ARMS)-PCR, and qualitative identification of the scented rice can be achieved by agarose gel electrophoresis or fluorescence quantitative PCR according to different laboratory conditions. Combined with internal reference primers for the calculation of gene level by fluorescence quantitative PCR method of scented rice and/or non-scented rice can achieve the adulteration detection of the scented rice quantitatively.

TECHNICAL FIELD

The disclosure relates to the field of biotechnologies, and more particularly to a qualitative and quantitative detection primer set, a kit and a detection method for scented rice.

### STATEMENT REGARDING SEQUENCE LISTING

The sequence listing associated with this application is provided in text format in lieu of a paper copy and is hereby incorporated by reference into the specification. The name of the XML file containing the sequence listing is 23063THXT-USP1-SL.xml. The XML file is 11,525 bytes; is created on Sep. 28, 2023; and is being submitted electronically via EFS-Web.

### BACKGROUND

Rice is an indispensable source of staple food in people's lives. Aroma is an important quality trait of high-quality grain. Scented rice is more favored by consumers because of its good aroma. Scented rice is soft, delicious, and nutritious, and is generally sold at a higher market price than non-scented rice. Aroma is not only the culinary quality of rice, but also the edible quality of rice. Rice aroma is a characteristic generally expected by rice consumers. Therefore, rice varieties with strong aromas have become a driving factor in consumers' preference for rice.

In view of the fact that in the process of grain purchase, some unscrupulous merchants, through fraud, adulterate non-scented rice with a similar appearance into scented rice for profiteering, and even add synthetic aromatic flavorings to create “artificially scented rice”. At present, the chewing method and the KOH method are the most commonly used methods for the traditional identification of scented rice, but these methods rely on sensory evaluation. The accuracy in identifying artificially added synthetic aromatic flavorings in rice is poor, and it is difficult to achieve quantitative detection of scented rice. 2-Acetyl-1-Pyrroline (2-AP) has been widely reported as a main contributor to the popcorn-like aroma of scented rice. Many studies at home and abroad have confirmed that the fgr gene (also referred to as OsBadh2 gene) on rice chromosome 8 controls its flavor, and the base deletion of the fgr gene leads to the production of truncated betaine aldehyde dehydrogenase 2 (BADH2) protein and promotes the production of 2-AP from its precurso. In this study, specific primers are designed for the base deletion site of exon 7, and the identification of scented rice at the gene level could be realized by using fluorescence quantitative polymerase chain reaction (PCR) gene technology.

From the perspective of detecting aroma genes of scented rice, a high-precision qualitative and quantitative technology for authenticity of scented rice is provided. It can be used to carry out actual commodity sampling and detection for main types of high-value scented rice in the market at present, such as typical scented rice like paddy aroma rice (also referred to as Daohuaxiang rice) and Thai Jasmine rice. The problem of low accuracy in identifying scented rice is solved, and the rapid and accurate qualitative identification of scented rice is realized, which has important application value in adulteration identification.

### SUMMARY

In order to solve the problems that in the process of grain purchase, some unscrupulous merchants, through fraud, adulterate non-scented rice with similar appearance into scented rice for profiteering, and even add synthetic aromatic flavorings to create “artificially scented rice”, a first purpose of the disclosure is to provide a qualitative and quantitative detection primer set for scented rice from the perspective of controlling the high-quality genes of rice aroma.

A second purpose of the disclosure is to provide a kit containing the primer set.

A third purpose of the disclosure is to provide a qualitative and quantitative detection method for the scented rice.

Technical solutions adopted by the disclosure to solve the technical problems are as follows.

In a first aspect, the disclosure provides a qualitative and quantitative detection primer set for scented rice, and the detection primer set includes one or more selected from the group consisting of five primers as follows:
internal reference primers:
   (01) ATGTCCATGCTGCAAGCAATGT, as shown in SEQ ID NO: 1;
   (02) GCACACCTGAAGGAAGACCAAC, as shown in SEQ ID NO: 2;
a general primer (forward):
   (03) ATCACACCCTGGTGTAGAC, as shown in SEQ ID NO: 3;
a scented rice primer (reverse):
   (04) CCATAGGAGCAGCTGAAATATA, as shown in SEQ ID NO: 4; and
a non-scented rice primer (reverse):
   (05) AGCAGCTGAAGCCATAATC, as shown in SEQ ID NO: 5.

The sequence 01 (forward primer, as shown in SEQ ID NO: 1) and the sequence 02 (reverse primer, as shown in SEQ ID NO: 2) are the internal reference sequences, especially the sequence 01 and sequence 02 are selected in a conservative region of the fgr gene (also referred to as OsBadh2 gene) to ensure that the amplification efficiency is not interfered by different varieties of rice. The sequence 03 (as shown in SEQ ID NO: 3) is the general forward primer. The sequence 04 (as shown in SEQ ID NO: 4) and the sequence 05 (as shown in SEQ ID NO: 5) are the reverse primers designed for specific gene loci of scented rice and non-scented rice, mainly to detect the fgr gene. Specifically, the sequence 04 is designed at the deletion of the fgr gene and spans two sides of the deletion site, and the sequence 05 is designed at the deletion site of the fgr gene and is matched with a deletion base.

In a second aspect, the disclosure provides an application of the primer set described above in the following (A1) or (A2):
(A1) an application in qualitative and quantitative analysis of scented rice;
(A2) an application in preparing qualitative and quantitative detection reagents or detection kits for scented rice.

In a third aspect, the disclosure provides a detection reagent and/or a detection kit, which includes the primer set described above.

In a specific embodiment, the detection kit further includes a 2× polymerase chain reaction (PCR) Master Mix (Vazyme, Nanjing, China, including Taq DNA Polymerase, dNTP and buffer system), a negative control and a positive control.

In a specific embodiment, the detection kit further includes a 2×SYBR qPCR Master Mix (Vazyme, Nanjing, China, including dNTP, Mg²⁺, Champagne Taq DNA Polymerase, SYBR Green I, Specific ROX Reference Dye), a negative control and a positive control.

In a specific embodiment, the negative control is a genomic DNA of a non-scented rice, such as Yueyou 938 (Fujian Appraised Rice 2006A01).

In a specific embodiment, the positive control is a genomic DNA of scented rice, such as Daohuaxiang 2 (Jilin Appraised Rice 2016011).

In a specific embodiment, a detection system is provided, and the detection system includes the detection reagent and/or the detection kit mentioned above.

In a fourth aspect, the disclosure requests to protect the application of the reagents and/or kits and/or detection systems mentioned above in the qualitative and quantitative analysis of scented rice.

In a fifth aspect, the disclosure provides a qualitative detection method of scented rice, PCR amplification is carried out by using the primer set mentioned above, and then 1% agarose gel electrophoresis detection is carried out, and the method includes the following steps:
step 1: extracting a genomic DNA of a sample;
step 2: preparing 20 μL reaction systems respectively for a scented rice primer and a non-scented rice primer, where the reaction system includes 0.1-1.0 microliter (μL) of a forward primer and a reverse primer with an initial concentration of 10 micromoles per liter (μM), 10 μL of 2×PCR Master Mix and 2 μL of template DNA, and double-distilled water (ddH₂O) with a volume supplemented to 20 μL;
step 3: performing PCR reaction, including:
   1) pre-denaturing at 95° C. for 30 seconds(s); and
   2) reacting at 95° C. for 10 s, performing amplification and extension reaction at 60-66° C. for 30 s, 30-40 cycles; and
step 4: performing 1% agarose gel electrophoresis for 15 minutes (min) under a constant pressure of 110-130 volts (V);
   when a PCR amplification product band appears in the reaction system corresponding to the scented rice primer, indicating that the sample contains the scented rice;
   when a PCR amplification product band appears in the reaction system corresponding to the non-scented rice primer, indicating that the sample contains the non-scented rice.

In a specific embodiment, the genomic DNA can be extracted by using a cetyltrimethylammonium bromide (CTAB) method, an alkaline extraction method, a sodium dodecyl sulfate (SDS) method, or a kit method.

In a specific embodiment, a cycling temperature during the PCR reaction is 63° C.

In a specific embodiment, the number of cycles in the PCR reaction is 35.

In a sixth aspect, the disclosure provides a quantitative detection method of scented rice by introducing internal reference primers, and detects the scented rice by fluorescence quantitative PCR after extracting DNA, which includes the following steps:
step 1: extracting a genomic DNA of a sample;
step 2: preparing 20 μL reaction systems for internal reference primers, a scented rice primer and a non-scented rice primer respectively, where the reaction system includes 0.1-1.0 μL of a forward primer and 0.2-0.4 μL of a reverse primer with an initial concentration of 10 μM, 2×SYBR qPCR Master Mix, and 2 μL of template DNA, and ddH₂O with a volume supplemented to 20 μL;
step 3: performing fluorescence quantitative PCR reaction, including:
   1) pre-denaturing at 95° C. for 30 s;
   2) reacting at 95° C. for 10 s, performing amplification and extension reaction at 60-66° C. for 30 s, 30-40 cycles, and collecting fluorescence signals after the extension reaction of each cycle is finished; and
   3) setting a melting curve program according to an instrument and an actual demand, where only parameters of ABI 7500 program setting are listed: reacting at 95° C. for 15 s, at 60° C. for 60 s, then rising to 95° C. at a rate of 1% for reacting 15 s, and different types of fluorescence quantitative PCR instruments can be set by default according to the instrument; and
step 4: reading a CT value of each reaction well after the reaction in the step 3 is completed, and calculating a scented rice proportion and a non-scented rice proportion respectively by using 2^{-ΔΔCT} method, so as to achieve the purpose of quantifying the scented rice in the sample.

In a specific embodiment, the genomic DNA can be extracted by using a CTAB method, an alkali extraction method, an SDS method, or a kit method.

In a specific embodiment, a cycling temperature during the PCR reaction is 63° C.

In a specific embodiment, the number of cycles in the PCR reaction is 35.

Beneficial effects of the disclosure are as follows.

The disclosure provides the qualitative and quantitative detection primer set, the kit and the detection method for the scented rice, which have the following advantages:
1. through the actual experimental verification, the adulteration ratio of the scented rice can be accurate within 10% deviation;
2. compared with the probe method, the reaction cost of common primers is greatly reduced; and
3. the whole reaction process is completed within 2 hours.

### BRIEF DESCRIPTION OF DRAWINGS

FIG. 1 illustrates difference sequence comparison of a fgr gene between a scented rice and a non-scented rice.

FIG. 2A illustrates positions of internal reference primers in genome.

FIG. 2B illustrates positions of scented rice primers in the genome.

FIG. 2C illustrates positions of non-scented rice primers in the genome.

FIG. 3A illustrates amplification efficiency of the internal reference primers, where a linear relationship of amplification efficiency of the internal reference primers is R²>0.99.

FIG. 3B illustrates amplification efficiency of the primers of the scented rice, where a linear relationship of amplification efficiency of the primers of the scented rice is R²>0.99.

FIG. 3C illustrates amplification efficiency of the primers of the non-scented rice, where a linear relationship of amplification efficiency of the primers of the non-scented rice is R₂>0.99.

FIG. 4A illustrates an amplification plot showing amplification curves of the primers of the scented rice.

FIG. 4B illustrates an amplification plot showing amplification curves of the primers of the non-scented rice.

FIG. 5 illustrates agarose gel electrophoresis results after polymerase chain reaction (PCR) amplification of the primers of the scented rice, where a lane 1 is DL 2000 base pairs (bp) marker, lanes 2-9 are PCR amplification products of Taiyou 553 (Guangxi Appraised Rice 2020025), Huiliangyou 719 (Nationally Appraised Rice 20200215), Huiliangyou 008 (Nationally Appraised Rice 20210160), Liangyou 9919 (Anhui Appraised Rice 2016037), Liaojing 305 (Liaoning Appraised Rice 2017007), Liaojing 327 (Liaoning Appraised Rice 2015017), Liaojing 169 (Liaoning Appraised Rice 2017027) and Yueyou 938 (Fujian Appraised Rice 2006A01) respectively, and lanes 10-12 are Daohuaxiang 2 (Jilin Appraised Rice 2016011), Nongxiang 24 (Hunan Appraised Rice 2016019) and Yuzhenxiang (Hunan Appraised Rice 2009038).

FIG. 6A illustrates a scented rice proportion determined by the primers of the scented rice, where the scented rice variety is Thai Jasmine rice and the non-scented rice variety is Yueyou 938. The scented rice proportion is set at 0%, 25%, 50%, 75%, 90% and 100% (w/w).

FIG. 6B illustrates a non-scented rice proportion determined by the primers of the non-scented rice, where the scented rice variety is Thai Jasmine rice and the non-scented rice variety is Yueyou 938. The scented rice proportion is set at 0%, 25%, 50%, 75%, 90% and 100% (w/w).

FIG. 7A illustrates a scented rice proportion determined by the primers of the scented rice, where the scented rice variety is Daohuaxiang 2 and the non-scented rice variety is Yueyou 938. The scented rice proportion of is set at 0%, 10%, 20%, 30%, 40%, 50%, 60%, 70%, 80%, 90% and 100% (w/w).

FIG. 7B illustrates a non-scented rice proportion determined by the primers of the non-scented rice, where the scented rice variety is Daohuaxiang 2 and the non-scented rice variety is Yueyou 938. The scented rice proportion of is set at 0%, 10%, 20%, 30%, 40%, 50%, 60%, 70%, 80%, 90% and 100% (w/w).

### DETAILED DESCRIPTION OF EMBODIMENTS

The disclosure will be further described in detail with reference to embodiments. The reagents or instruments used are not indicated by the manufacturer, they are regarded as conventional products that can be purchased through the market.

### Embodiment 1 Qualitative Detection of Scented Rice

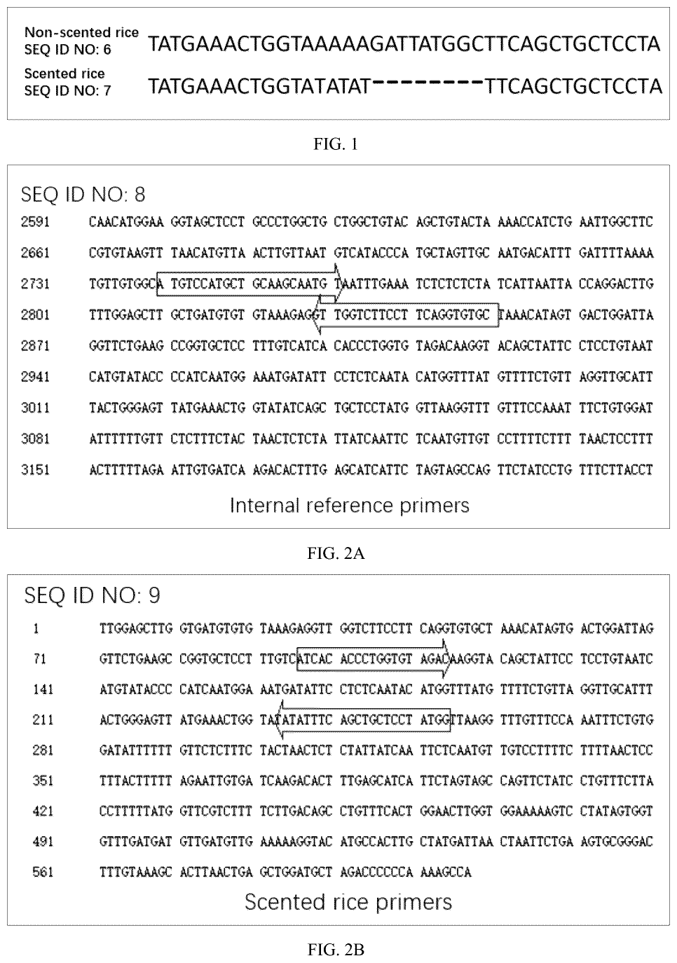

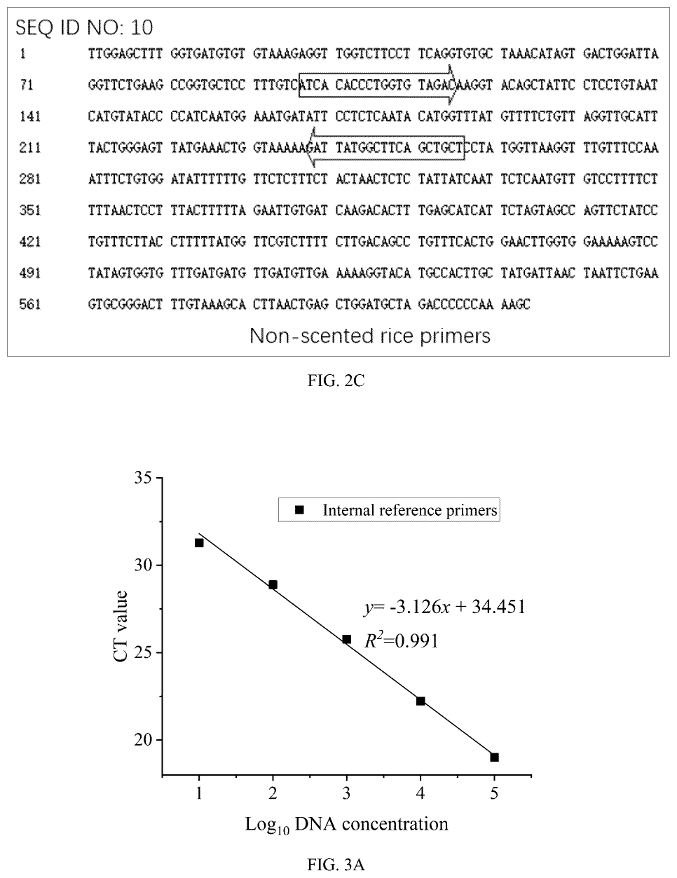

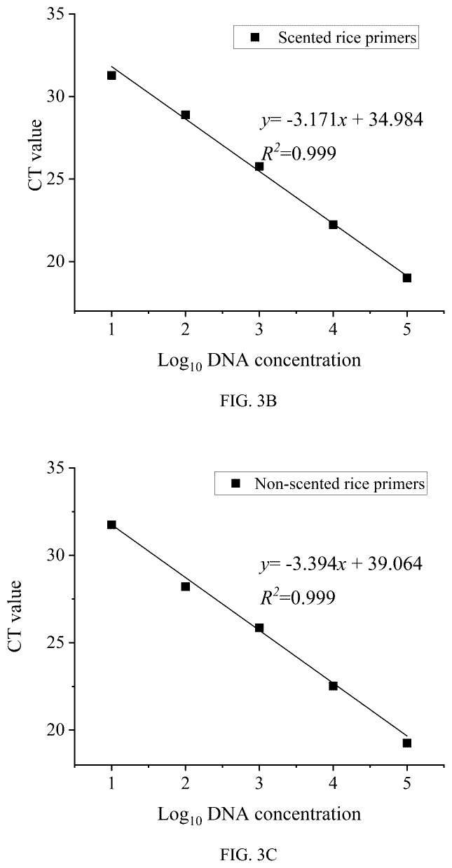

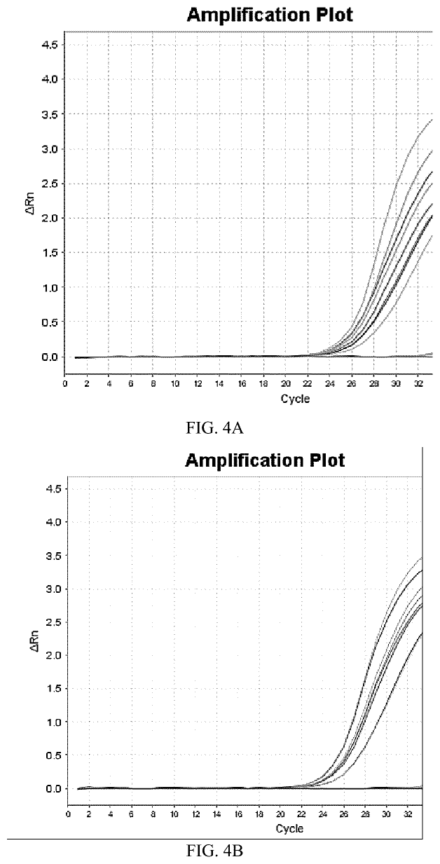

1. Rice samples: 8 kinds of non-scented rice varieties, namely Taiyou 553 (Guangxi Appraised Rice 2020025), Huiliangyou 719 (Nationally Appraised Rice 20200215), Huiliangyou 008 (Nationally Appraised Rice 20210160), Liangyou 9919 (Anhui Appraised Rice 2016037), Liaojing 305 (Liaoning Appraised Rice 2017007), Liaojing 327 (Liaoning Appraised Rice 2015017), Liaojing 169 (Liaoning Appraised Rice 2017027) and Yueyou 938(Fujian Appraised Rice 2006A01); and 3 kinds of scented rice varieties, namely Daohuaxiang 2 (Jilin Appraised Rice 2016011), Nongxiang 24 (Hunan Appraised Rice 2016019) and Yuzhenxiang (Hunan Appraised Rice 2009038). All of the scented and non-scented rice cultivars was classified by gene sequencing (as shown in FIG. 1).

2. Method of extracting genomic DNA from samples

In this embodiment, the improved cetyltrimethylammonium bromide (CTAB) method is used to extract rice genomic DNA, specifically includes steps as follows.
Step (1): 20-50 grams (g) rice samples are weighed and pulverized with a grinder to obtain pulverized samples. 50 milligrams (mg) the pulverized samples are weighed and put into a 1.5 milliliters (mL) centrifuge tube, 500 microliters (μL) CTAB extracting solution are added into the centrifuge tube, and shaken up and down evenly.
Step (2): 500 μL chloroform-isoamyl alcohol (24:1) are added into the centrifuge tube and centrifuged at 12,000 g for 5 minutes (min) after shaking evenly to obtain a first supernatant.
Step (3): 400 μL of the first supernatant obtained in the step (2) is transferred to another 1.5 mL centrifuge tube, 800 μL of ethanol precooled at −20° C. is added to precipitate DNA, and then centrifuged for 5 min at 12000 g, so as to obtain a second supernatant and a precipitate.
Step (4): The second supernatant obtained in the step (3) is discarded, and then the precipitate is rinsed twice with 70% ethanol solution.
Step (5): The rinsed precipitate is dried under natural conditions to obtain a dried product, 100 μL ddH₂O is added to the dried product to dissolve DNA and detect the concentration.

3. PCR reaction system: 20 μL reaction systems are prepared for primers of scented rice and non-scented rice respectively, the 20 μL reaction system includes 1.0 μL forward primer and 1.0 μL reverse primer with an initial concentration of 10 micromoles per liter (μM), 10 μL of 2×PCR Master Mix, 2 μL of template DNA, and ddH₂O with a volume supplemented to 20 μL.

4. PCR reaction is performed as follows:
step (i): pre-denaturing at 95° C. for 30 seconds(s); and
step (ii): reacting at 95° C. for 10 s, performing amplification and extension reaction at 65° C. for 30 s, 30 cycles.

5. Under the constant pressure of 110-130 volts (V), 1% agarose gel electrophoresis is performed for 15 min. When a PCR amplification product band appears in the reaction system corresponding to the primers of the scented rice, it is indicated that the sample contains the scented rice; and when a PCR amplification product band appear in the reaction system corresponding to the primers of the non-scented rice, it is indicated that the sample contains the non-scented rice.

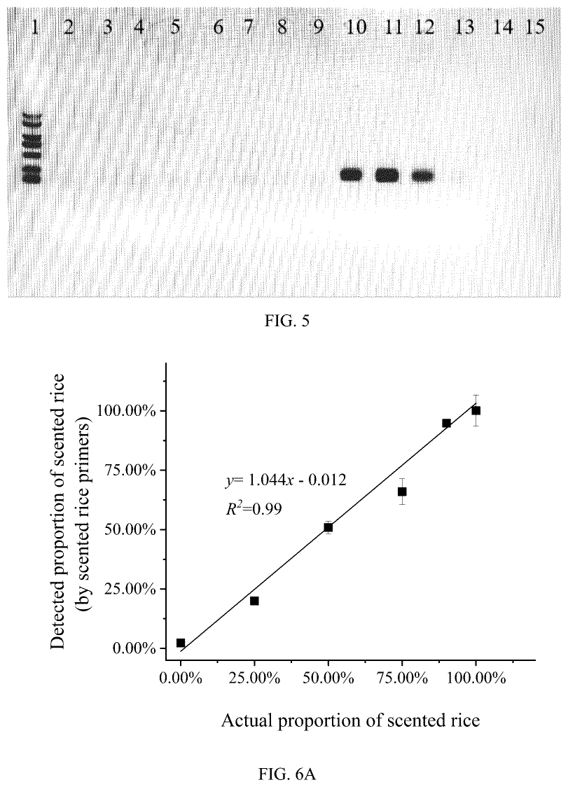

Results are as shown in FIG. 5, lane 1 is DL 2000 base pairs (bp) marker, lanes 2-9 are PCR amplification products of Taiyou 553, Huiliangyou 719, Huiliangyou 008, Liangyou 9919, Liaojing 305, Liaojing 327, Liaojing 169 and Yueyou 938 respectively, and lanes 10-12 are Daohuaxiang 2 and Nongxiang 24. The results show that Taiyou 553, Huiliangyou 719, Huiliangyou 008, Liangyou 9919, Liaojing 305, Liaojing 327, Liaojing 169 and Yueyou 938 are non-scented rice, while Daohuaxiang 2, Nongxiang 24 and Yuzhenxiang are scented rice.

### Embodiment 2 Quantitative Detection of Scented Rice

1. Rice samples: artificially adulterated scented rice samples with different proportions, including 0%, 25%, 50%, 75% and 90% (w/w) scented rice, in which the scented rice is Thai jasmine rice and the non-scented rice is Yueyou 938.

2. Method of extracting rice genomic DNA from samples

In this embodiment, the improved sodium dodecyl sulfate (SDS) method is used to extract rice genomic DNA, specifically includes steps as follows.
Step (1): 20-50 g rice samples are weighed and pulverized with a grinder to obtain pulverized samples. 50 mg the pulverized samples are weighed and put into a 1.5 mL centrifuge tube, 500 μL SDS extracting solution are added into the centrifuge tube, and shaken up and down evenly.
Step (2): 500 μL chloroform-isoamyl alcohol (24:1) is added into the centrifuge tube and centrifuged at 10,000 g for 5 min after shaking evenly to obtain a first supernatant.
Step (3): 400 μL of the first supernatant obtained in the step (2) is transferred to another 1.5 mL centrifuge tube, 800 μL of ethanol precooled at −20° C. is added to precipitate DNA, and then centrifuged for 5 min at 10,000 g, so as to obtain a second supernatant and a precipitate.
Step (4): The second supernatant obtained in the step (3) is discarded, and then the precipitate is rinsed twice with 70% ethanol solution.
Step (5): The rinsed precipitate is dried under natural conditions to obtain a dried product, 100 μL ddH₂O is added to the dried product to dissolve DNA and detect the concentration.

3. Preparation of PCR reaction systems: 20 μL reaction systems are prepared for internal reference primers and primers of scented rice and non-scented rice respectively. The 20 μL reaction system includes 0.4 μL forward primer and 0.4 μL reverse primer with an initial concentration of 10 μM, 10 μL of 2×SYBR qPCR Master Mix, 2 μL of template DNA, and ddH₂O with a volume supplemented to 20 μL.

4. Fluorescence quantitative PCR reaction is performed under the following conditions:
step (i): pre-denaturing at 95° C. for 30 s;
step (ii): reacting at 95° C. for 10 s, performing amplification and extension reaction at 60° C. for 30 s, totally 40 cycles, and collecting fluorescence signals after the extension reaction of each cycle is finished; and
step (iii): setting a melting curve program according to an instrument and an actual demand, where only parameters of ABI 7500 program setting are listed: reacting at 95° C. for 15 s, at 60° C. for 60 s, then rising to 95° C. at a rate of 1% for 15 s.

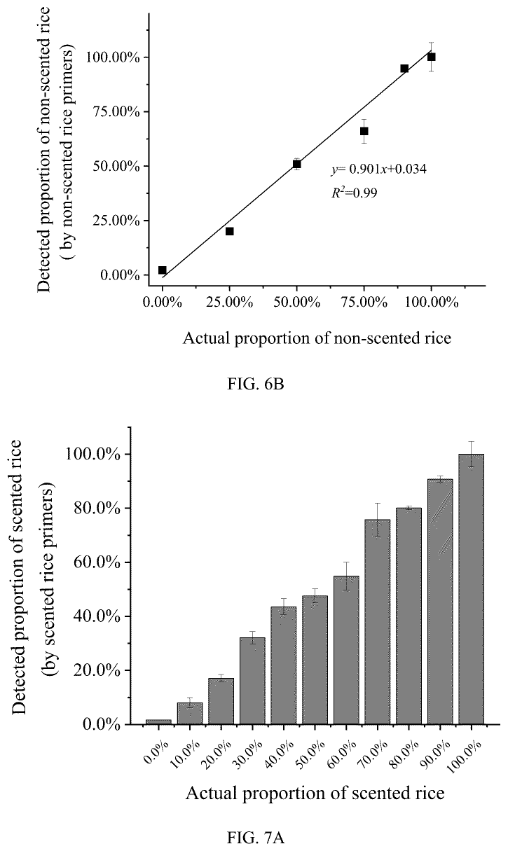

5. A CT value of each reaction well is read, and the scented rice proportion and non-scented rice proportion are calculated by using 2^{-ΔΔCT} method respectively, so as to achieve the quantitative purpose of the scented rice in the samples. The results are shown in FIG. 6A and FIG. 6B.

### Embodiment 3 Quantitative Detection of Scented Rice

1. Rice samples: artificially adulterated scented rice samples with different proportions, in which the scented rice variety is Daohuaxiang 2, the non-scented rice variety is Yueyou 938,and the proportion of scented rice is set at 0%, 10%, 20%, 30%, 40%, 50%, 60%, 70%, 80%, 90% and 100% (w/w).

2. Method of extracting rice genomic DNA from samples

In this embodiment, the DNA is extracted from the samples by using a plant genomic DNA extraction kit (Nanjing Vazyme Biotech Co., Ltd.).

3. 20 μL reaction systems are prepared for internal reference primers and primers of scented rice and non-scented rice respectively. The 20 μL reaction system includes 0.2 μL forward primer and 0.2 μL reverse primer with an initial concentration of 10 μM, 10 μL of 2×SYBR qPCR Master Mix, 2 μL of template DNA, and ddH₂O with a volume supplemented to 20 μL.

4. Fluorescence quantitative PCR reaction is performed under the following conditions:
step (i): pre-denaturing at 95° C. for 30 s;
step (ii): reacting at 95° C. for 10 s, performing amplification and extension reaction at 63° C. for 30 s, totally 35 cycles, and collecting fluorescence signals after the extension reaction of each cycle is finished; and
step (iii): setting a melting curve program according to an instrument and an actual demand, where only parameters of ABI 7500 program setting are listed: reacting at 95° C. for 15 s, at 60° C. for 60 s, then rising to 95° C. at a rate of 1% for 15 s.

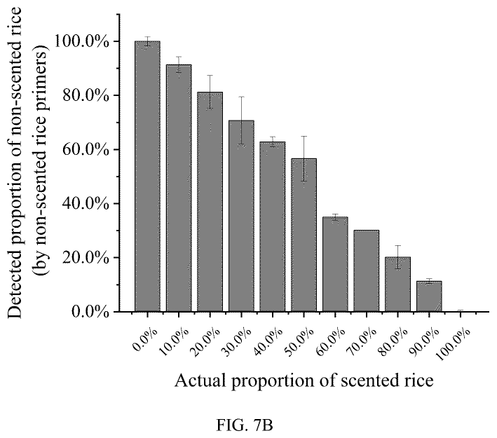

5. A CT value of each reaction well is read, and the scented rice proportion and non-scented rice proportion are calculated by using 2^{-ΔΔCT} method respectively, so as to achieve the quantitative purpose of the scented rice in the samples. The results are shown in FIG. 7A and FIG. 7B.

The protection content of the disclosure is not limited to the above embodiments. Without departing from the spirit and scope of the inventive concept, variations and advantages that can be thought of by those skilled in the art are included in the disclosure, and the scope of protection is the appended claims.

What is claimed is:

1. A primer set, comprising two or more selected from the group consisting of sequences of SEQ ID NOs: 1-5.

2. The primer set as claimed in claim 1, wherein the primer set is applied for qualitative and quantitative analysis of scented rice; or the primer set is applied for preparing one of a qualitative and quantitative detection reagent and a qualitative and quantitative detection kit for the scented rice.

3. A detection reagent, comprising the primer set as claimed in claim 1.

4. A detection kit, comprising the primer set as claimed in claim 1.

5. The detection kit as claimed in claim 4, further comprising a 2× polymerase chain reaction (PCR) Master Mix, a negative control and a positive control.

6. The detection kit as claimed in claim 4, further comprising a 2×SYBR quantitative polymerase chain reaction (qPCR) Master Mix, a negative control and a positive control.

7. The detection kit as claimed in claim 5, wherein the negative control is a genomic DNA of non-scented rice, and the non-scented rice is Yueyou 938.

8. The detection kit as claimed in claim 6, wherein the negative control is a genomic DNA of non-scented rice, and the non-scented rice is Yueyou 938.

9. The detection kit as claimed in claim 5, wherein the positive control is a genomic DNA of scented rice, and the scented rice is Daohuaxiang 2.

10. The detection kit as claimed in claim 6, wherein the positive control is a genomic DNA of scented rice, and the scented rice is Daohuaxiang 2.

11. An application method of the detection kit as claimed in claim 4, comprising:
performing qualitative and quantitative analysis on scented rice by using the detection kit.

12. A qualitative detection method of scented rice, using the primer set as claimed in claim 1 for detection, comprising the following steps:
step 1: extracting a genomic DNA of a sample;
step 2: preparing 20 μL reaction systems respectively for a scented rice primer and a non-scented rice primer, wherein the reaction system comprises 0.1-1.0 microliter (μL) of a forward primer and a reverse primer with an initial concentration of 10 micromoles per liter (μM), 10 μL of 2×PCR Master Mix, 2 μL of template DNA, and ddH₂O with a volume supplemented to 20 μL;
step 3: performing a PCR reaction with the genomic DNA of the sample and the reaction system, comprising:
1) pre-denaturing at 95° C. for 30 seconds(s); and
2) reacting at 95° C. for 10 s, performing amplification and extension reaction at 60-66° C. for 30 s, 30-40 cycles; and
step 4: performing 1% agarose gel electrophoresis for 15 minutes (min) under a constant pressure of 110-130 volts (V) on the sample after the PCR reaction, and when a PCR amplification product band appears in the reaction system corresponding to the scented rice primer, indicating that the sample contains the scented rice; when a PCR amplification product band appears in the reaction system corresponding to the non-scented rice primer, indicating that the sample contains non-scented rice.

13. A quantitative detection method of scented rice, using the primer set as claimed in claim 1 for detection, comprising the following steps:
step 1: extracting a genomic DNA of a sample;
step 2: preparing 20 μL reaction systems for internal reference primers, a scented rice primer and a non-scented rice primer respectively, wherein the reaction system comprises 0.1-1.0 μL of a forward primer and 0.2-0.4 μL of a reverse primer with an initial concentration of 10 μM, 2×SYBR qPCR Master Mix, and 2 μL of template DNA, and ddH₂O with a volume supplemented to 20 μL;
step 3: performing fluorescence quantitative PCR reaction with the genomic DNA of the sample and the reaction system, comprising:
1) pre-denaturing at 95° C. for 30 s;
2) reacting at 95° C. for 10 s, performing amplification and extension reaction at 60-66° C. for 30 s, 30-40 cycles, and collecting fluorescence signals after the extension reaction of each cycle is finished; and
3) reacting at 95° C. for 15 s, at 60° C. for 60 s, then rising to 95° C. at a rate of 1% for reacting 15 s; and
step 4: reading a CT value of each reaction well after the reaction in the step 3 is completed, and calculating a scented rice proportion and a non-scented rice proportion respectively by using 2^{-ΔΔCT} method, so as to achieve quantitative detection of the scented rice in the sample.
